(19) United States
(12) Patent Application Publication
(10) Pub. No.: US 2003/0003568 A1
(43) Pub. Date: Jan. 2, 2003

(54) Waste management by source

(76) Inventor: Gursel G. Yilmaz, Bonita Springs, FL (US)

Correspondence Address: Gursel G Yilmaz, 28202 Meadowlark Lane, Bonita Springs, FL, 34134, US

(21) Appl. No.: 09/576,877

(22) Filed: May 22, 2000

(51) Int. Cl. C12M 1/00; B27J 1/00; B27K 7/00

(52) U.S. Cl. 435/262; 209/930; 241/28

(57) ABSTRACT

Waste management has been primarily based on collection of waste and placing most of it in a landfill. Waste management by source eliminates a need for large landfills by separating waste into wet organic waste and dry waste and processing said waste for reuse prior to landfill application. Waste management by source integrates collection and transportation of waste with separation, treatment, processing, and recovery using a collection apparatus, one or a combination of hydrotransportation system and a transportation vehicle, a wet organic waste processing plant, and a dry waste processing facility for reuse. Since untreated organic waste is eliminated or significantly reduced from waste stream, present invention eliminates a need for daily cover and working front of landfills resulting in elimination of extensive leachate management systems and associated potential groundwater and air pollution problems.

### FIELD OF THE INVENTION

The present invention lies in the field of civil engineering and more particularly in waste management including collection, transportation, and processing of waste for reuse.

### BACKGROUND OF THE INVENTION

Waste management commonly called municipal or solid waste management has been primarily based on collection of waste and placing most if not all of it in a landfill. Present invention eliminates a need for large landfills by using a concatenated collection apparatus, a transportation vehicle, and a processing facility for reuse. Using present invention current and future landfill sites are converted into a waste processing and treatment facility which includes controlled biodegration and eliminates open working front of a landfill operation and all associated odor and leachate problems. Voluminous prior art reviewed showed improving large landfills and waste management by means of landfill reclamation, landfill mining, landfill bioreactors and recovery. However, prior art reviewed did not demonstrate a novel approach that will interconnect and integrate collection, transportation, and processing of waste. Relevant examples of prior art on processing and disposal of waste management are summarized below.

U.S. Pat. No. 3,635,409 discloses a system and method for organic and inorganic municipal waste processing for reuse including crushing garbage and separating garbage after its crushed.

U.S. Pat. No. 1,329,105 discloses an apparatus for waste disposal and treatment in tower like structures having a number of chambers which air conduits extent vertically through said chambers.

U.S. Pat. No. 1,832,179 discloses treatment of organic refuse into useful substances by injecting air into moistened refuse.

U.S. Pat. No. 2,798,800 discloses a process which includes windrow referred as pile of unsegregated municipal refuse. The windrow is tumbled to provide necessary oxygen within said windrow to support aerobic process as needed.

U.S. Pat. No. 3,298,821 discloses a method and apparatus for decomposing waste material by aerobic process which is promoted and optimized by conditions designed for aerobic bacterial activity.

U.S. Pat. No. 3,419,377 discloses a method for treating organic and inorganic waste material. Said material is pulverized, mixed, and moistened to start fermentation prior to a digester chamber.

U.S. Pat. No. 4,844,813 discloses a system and process for treatment of biodegradable waste which includes a land treatment area underlain by an impermeable layer and surrounded by dikes. A leachate collection system permits effluent collection and routes said effluent to a wastewater treatment system.

U.S. Pat. No. 4,543,016 discloses underground leachate barrier and method which includes digging a trench adjacent a contaminated area, placing a liquid impervious membrane on one side of said trench, and positioning drain pipe and risers surrounded by filter gravel within said trench.

U.S. Pat. No. 5,078,882 discloses bioconversion reactor and system which is claimed to be useful for the biological transformation of waste material into ecologically desirable materials. Said system is referred and defined as a group of zones including bioreactor zone, solids ecoreactor zone, georeactor zone, all of which said zones are interconnected. Said system includes wetlands, marshes, wastes land filled under soil like material with marsh plants.

U.S. Pat. No. 5,201,609 discloses cellular landfill process and apparatus wherein waste are disposed of in a landfill repository that maintains them in a dry state indefinitely using water and gas tight cells.

U.S. Pat. No. 5,265,979 discloses a high efficiency waste placement system for municipal landfills which includes shredding the waste, adjusting the moisture of the waste, installing an aeration system in a configured pile of said waste, covering the pile for aerobic decomposition, compacting the waste pile to be covered with a synthetic cover.

U.S. Pat. No. 5,348,422 discloses method for the formation and operation of in situ process reactor using a mobile trenching machine which converts a contaminated site to a reactor by simultaneously placing contaminant impermeable walls while processing excavated materials such as adding reactor reagents.

U.S. Pat. No. 5,356,452 discloses a method and apparatus for reclaiming waste materials. Waste materials are placed over impermeable liner in a domed structure. The decomposition of the waste material is controlled and monitored and after a period of time, the material within one or more cells is recovered and recycled.

U.S. Pat. No. 5,429,454 discloses a method for landfill reclamation which primarily includes excavation of waste materials from a landfill, separation of excavated waste materials, recovery of recyclable from excavated waste materials, and placing unrecoverable excavated waste materials back into the landfill.

U.S. Pat. No. 5,564,862 discloses a method of improved landfill mining which comprises converting the landfill to aerobic production by injection of air, moisture, and sludge for increased rate of decomposition, and excavating the landfill to remove waste materials, separating the removed waste material, and returning the residual to the landfill.

Prior art reviewed as summarized above does not demonstrate a new and novel concatenated approach to waste management by source including a waste collection apparatus, one or a combination of a waste transportation vehicle and a hydrotransportation system, and a waste processing facility for reuse. Approach to waste management by source interconnects and integrates collection and transportation of waste with treatment, processing, recovery, and reuse of waste using concatenated waste collection apparatus, one or a combination of a waste transportation vehicle and waste hydrotransportation system, and a waste processing facility. As a result, the waste is converted into two primary groups of wet organic waste and dry waste most of which can be processed for reuse prior to landfill application. Therefore, present invention eliminates a need for permanently placed large landfills which become a continuous environmental, economical, and public health threat to the surrounding communities. Although it is preferred to eliminate a need for large landfill sites, present invention may be used in association with or as a part of a small or large landfill site depending on the waste stream and market conditions. One of the primary benefits of using present invention is the elimination of open working front of a landfill operation and all associated odor and leachate problems related to wet organic waste portion of waste.

### SUMMARY OF THE INVENTION

Waste management has been primarily based on collection of waste and placing it in a landfill which has to be continuously monitored and maintained. Waste management by source eliminates a need for large landfills. Waste management by source integrates collection and transportation of waste with separation, treatment, processing, recovery, and reuse of waste. Current and future landfill sites can be primarily used for waste treatment and processing which eliminates open working front of a landfill operation and all associated odor and leachate problems. Waste is preferred to be separated into two primary groups of wet organic waste and dry waste most of which can be processed and recovered before it goes to a landfill. Therefore, present invention eliminates a need for permanently placed large landfills which become a continuous environmental, economical, and social threat to the surrounding communities.

Prior art reviewed demonstrated that a number of methods and apparatus which improved a concept of large landfills which consist of burying waste under controlled environmental conditions and long term monitoring of said conditions.

However, prior art reviewed does not demonstrate a new comprehensive novel facility and utility system which will eliminate a concept of large landfills. Present invention makes collection and transportation of waste, a significant part of treatment, processing, recovery, and reuse prior to landfill application. A portion of said waste which is not recovered and reused is disposed in a small landfill or stored for further processing.

Waste is collected and transported as dry waste and wet organic waste most of which are separated at source and initially prepared for processing or treatment during the transportation. Prior to landfilling, said dry waste and wet organic waste are accepted by dry recovery process and wet recovery process respectively. Dry recovery process includes recovery and reuse of cellulose based materials such as paper, plastics such as high and low density plastics, yard waste such as grass clippings and trees, construction demolition materials such as concrete and iron bars, rubber and petroleum product material such as tires and geotextile. Wet recovery process includes recovery and reuse of mostly household kitchen waste, sludge from household septic tanks or other sources, and other wet organic waste such as waste from food and drink processing facilities with high BOD demand such as restaurants and hotels. Since most of the waste is processed and prepared for reuse prior to landfilling using present invention, only a small portion of waste is left for landfilling or further processing for reuse. Although it is preferred to eliminate a need for large landfill sites, present invention may be used in association with or as a part of a small or large landfill site depending on the waste stream and market conditions. One of the primary benefits of using present invention is elimination of open working front of a landfill operation and all associated odor and leachate problems related to wet organic waste portion of waste. The following is a partial list of benefits of the present invention through which

organic waste is separated and processed for reuse and is not buried as untreated wet organic waste in a landfill or bioreactor, and therefore

a large quantities of daily cover material are not needed resulting in cost reductions in operations associated with landfill and waste management,

a working front of a landfill is eliminated or significantly reduced resulting in elimination of odor and associated local political considerations such as relocation of existing landfills at high costs to community at large,

a need for an extensive leachate management system for organic compounds is eliminated resulting in reduction of potential groundwater and surface water pollution and associated environmental liabilities,

a need for extensive gas management systems is also eliminated resulting in air pollution and associated cost reductions,

a need for expensive and extensive curbside recycling is eliminated resulting in opportunities for more frequent waste collections with less complicated collection schedules. However, present inventions allows such recycling programs where it is found to be cost effective.

### BRIEF DESCRIPTION OF THE DRAWINGS

FIG. 1.1 shows a process chart of the waste hydrotransportation system and collection unit including a wet waste lift station and a wet waste transfer station and wet waste generator collection apparatus and wet organic waste processing plant.

FIG. 1.2 shows a process chart of the present invention including generator, wet organic waste and dry waste generator containers, a transportation and preparation vehicle or unit, and wet organic waste processing plant and dry waste processing facility.

FIG. 2 shows a top view of a transportation and preparation vehicle or unit.

FIG. 3 shows a cross section view of a transportation and preparation vehicle or unit.

FIG. 4 shows a plan view of the present invention including wet organic waste processing plant and storage of wet organic waste for reuse.

FIG. 5 shows a processing chart of the present invention including dry waste processing facility and storage of dry waste for reuse.

FIG. 6 shows construction waste processing unit or plant including separation and decontamination and treatment of construction and demolition waste portion of the waste before reuse.

FIG. 7 shows combined dry waste processing unit or plant including separation and decontamination and treatment of combined dry waste portion of the waste before reuse.

FIG. 8 shows elastic waste processing unit or plant including separation and decontamination and treatment of elastic waste portion of the waste before reuse.

FIG. 9 shows botanical waste processing unit or plant including separation and decontamination and treatment of botanical waste portion of the waste before reuse.

### DETAILED DESCRIPTION OF THE PREFERRED EMBODIMENTS

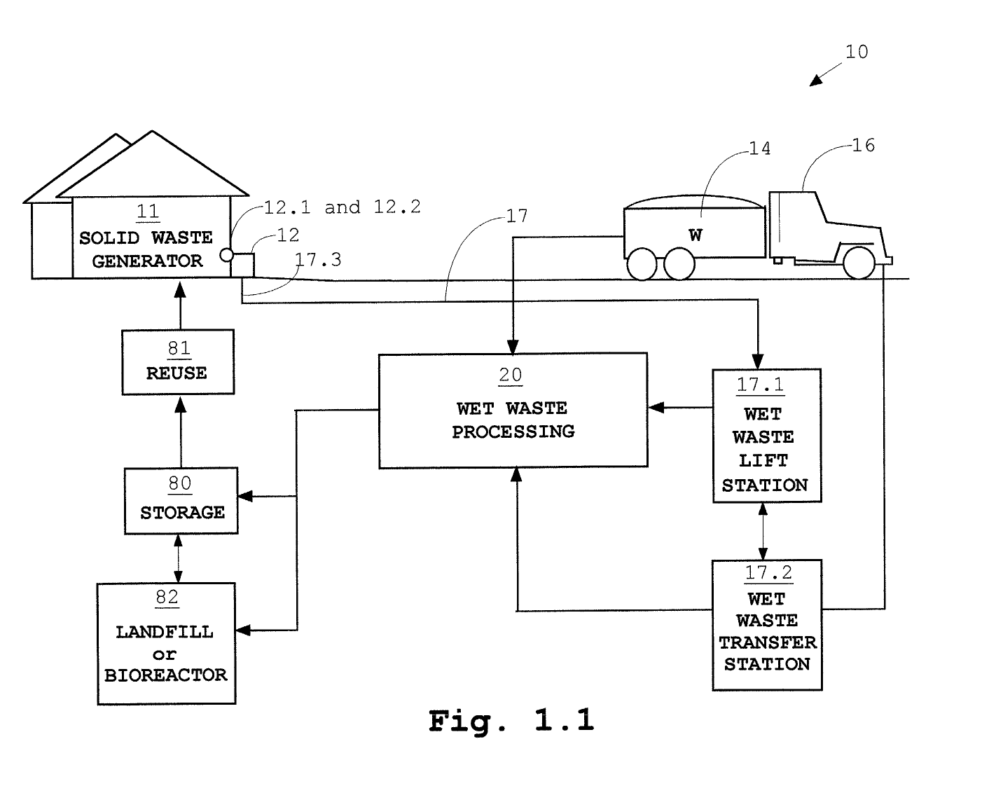

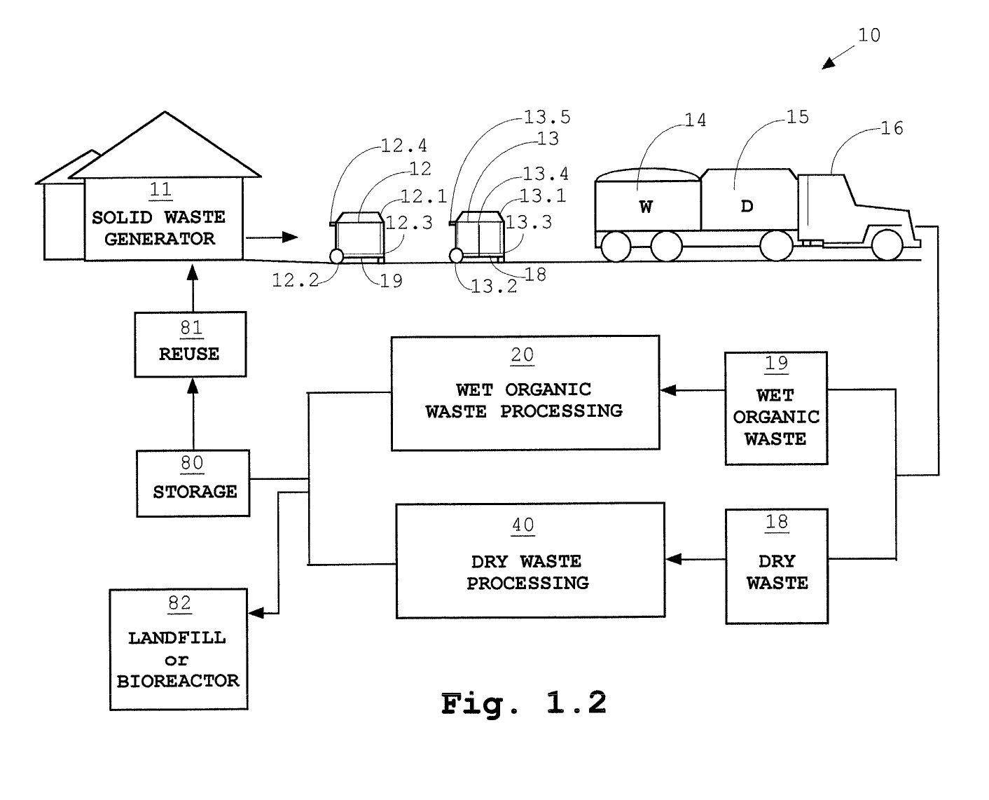

FIG. 1.1 shows hydrotransportation of waste collection and processing system 10 including a waste generator 11, preferred wet waste generator collection apparatus 12 and waste hydrotransportation system 17. Waste commonly called municipal or solid waste is transported using system 17. Said hydrotransportation system 17 includes a collection unit consisting of one or a combination of collection apparatus 12 and wet waste lift station 17.1 and wet waste transfer station 17.2, and a transportation pipe/main 17.3. Said collection unit is designed to add water and reduce said wet waste in size for hydrotrasportation using one or a combination of a generator grinder and generator cutter. Collection apparatus 12 is preferred to add water and reduce said wet waste in size for hydrotransportation using one or a combination of a generator grinder 12.1 and generator cutter 12.2.A. A wet waste processing plant 20 receives wet waste from one or a combination of wet waste lift station 17.1 and wet waste transfer station 17.2 and wet waste transportation vehicle/unit 16. Wet waste is prepared by one or a combination of wet waste generator collection apparatus 12 and wet waste lift station 17.1 and wet waste transfer station 17.2 and wet waste transportation vehicle/unit 16 for hydrotransportation system 17. Wet waste 19 is processed using wet waste plant 20 which uses one or a combination of aerobic and anaerobic methods for storage 80 and reuse 81. Any waste which can not be cost effectively processed for reuse is stored or deposited in a small landfill or bioreactor 82.

FIG. 1.2 shows interconnected waste collection and transportation and processing system 10 including summary of the present invention showing a waste generator 11, preferred waste collection apparatus and sub-system including wet organic waste generator container 19 and dry waste generator container 18 for separation of waste into groups of at least wet organic waste 19 and dry waste 18 as initially contained in a wet organic waste generator container 12 and a dry waste generator containers 13 respectively, a transportation and preparation vehicle/unit and sub-system 16, wet organic waste processing plant 20, and dry waste processing facility 40. Wet organic waste generator container 12 includes a cover 12.1 and at least one wheel 12.2 and handle 12.4 and a biodegradable removable liner 12.3 which covers the inside wall of said wet organic waste generator container 12. Dry waste generator container 13 includes a cover 13.1 and handle 13.5 and at least one wheel 13.2 and removable liner 13.3 which covers the inside wall of said dry waste generator container 13 and an adjustable partitioning system 13.4 for different groups of dry waste portion of the waste. Said transportation and preparation unit 16 is preferred to have two transportation containers 14 and 15 for wet organic waste 19 and dry waste 18 respectively for the purpose of preparation and initial processing. Wet organic waste 19 is prepared through processing plant 20 for reuse 81 and storage 80. Dry waste 18 is prepared through processing 40 for storage 80 and reuse 81. Any waste which can not be cost effectively processed for reuse is stored or deposited in a small landfill or bioreactor 82.

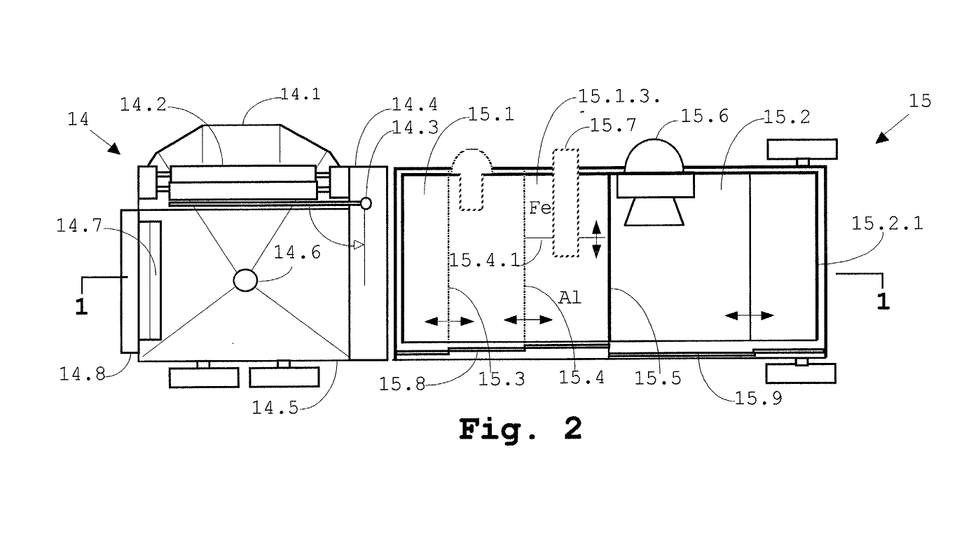

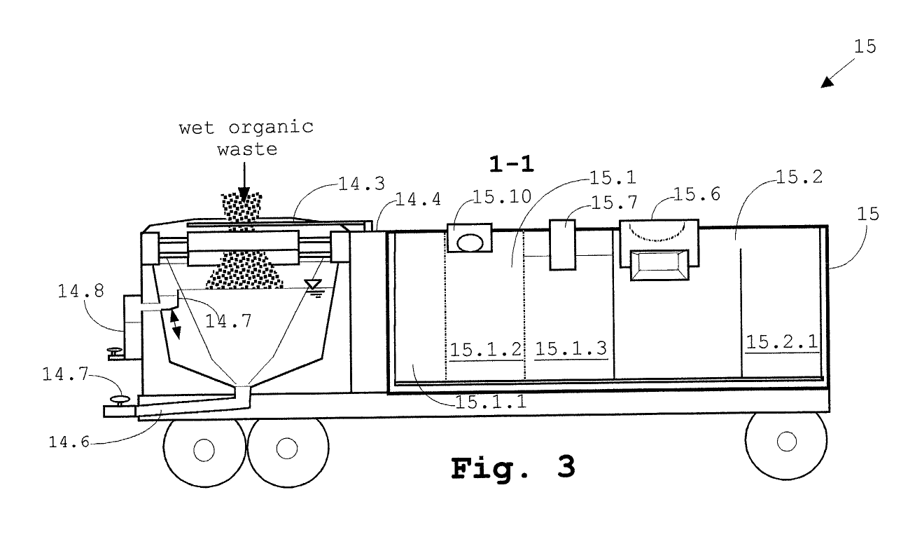

FIG. 2 and FIG. 3 shows a top view and a cross section of a transportation and preparation vehicle/unit and sub-system 16 which includes wet organic waste and dry waste transportation vehicles or units that have containers 14 and 15 respectively. Wet organic waste transportation container 14 and dry waste transportation containers 15 receives waste from wet organic waste generator container 12 and dry waste generator container 13 respectively. Preparation and initial processing of wet organic waste 19 is primarily achieved during collection and transportation as follows. Wet organic waste 19 is accepted through feeder 14.1 and prepared by transportation grinder 14.2 while being watered by water spray nozzle 14.3 which is connected to water tank 14.4. Transportation watering unit 14.3 is turned on and positioned as needed. Said waste 19 is contained in 14.5 which can also be used to separate grease and discharged using 14.6 and valve 14.7.

Preparation and initial processing of dry waste 18 is primarily achieved using wet organic sub-system and dry waste sub-system during collection and transportation as follows. Dry waste 18 is separated as much as possible using generator container 13 and dry waste transportation container 15. Dry waste transportation container 15 consist of one section 15.1 or multiple sections 15.5., 15.6, 15.7, 15.8 using adjustable partitions 15.2, 15.3, and 15.4 respectively for the purpose of segregating dry waste as a function of waste stream and transporting said waste for processing 40. Although preferred to be segregated where cost effective, dry waste 18 can be collected and transported without segregation as a combined dry waste using 15.1. Dry waste processing 40 would segregate said combined dry waste for reuse using 15.1 for transportation and 40 for processing. Local conditions and characteristics of the waste stream would be one of the primary selection criteria for above mentioned options of combined or segregated dry waste collection and transportation.

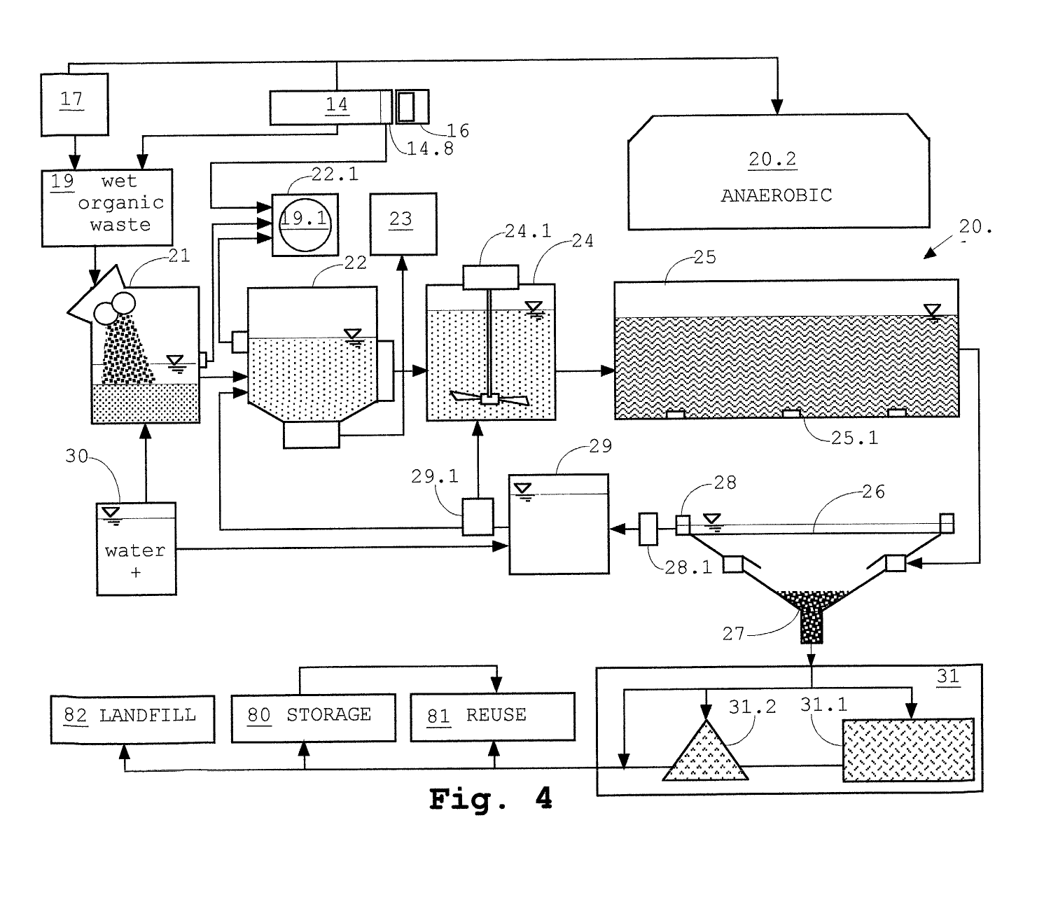

FIG. 4 shows waste processing plant and sub-system 20 for wet organic waste portion of waste including a wet organic waste processing facility and plant 20 which utilize one or a combination of aerobic 20.1 and anaerobic 20.2 methods for organic waste reduction. The following is presented as the preferred aerobic plant 20.1. Wet organic waste 19 from 14 is accepted by grinding unit 21 to which fluid mostly water is added from a primary fluid tank 30. Wet organic waste 19 is mixed with additional fluid from a secondary fluid tank 29 and is separated by density using density filtering unit 22. Said waste 19 is separated and filtered as a function of density using unit 22 and waste such as grease 19.1, ferrous, aluminum, plastic are as much as possible separated from wet organic waste 19 and transported to dryer unit 23 and then accepted by dry waste processing 40. Grease 19.1 is also collected from 21 as desired. Remaining wet organic waste is accepted by mixing unit 24 where additional fluid mostly water from secondary fluid tank 29 is added and thoroughly mixed then accepted by aerating unit 25 where rapid organic waste reduction is achieved. A settling unit 26 receives wet organic waste and fluid mostly water from 25 and settles organic solids in 27 and clarifies fluid in 28 and collects the fluid in secondary fluid tank 29. Settled organic solids in 27 is received by conditioning unit 31 for reuse such as soil conditioning products and mulch. Said conditioning unit 31 includes dewatering unit 31.1 such as drying beds and wet organic compost unit 31.2. and utilized as needed for processing said settled organic solids for reuse 81 and stored in storage unit 80 or placed in a landfill 82 as needed. In summary, preferred waste processing sub-system for wet organic waste portions of said waste includes a wet organic waste processing plant comprising

a wet organic waste generator container 19 for collection,

a transportation and preparation vehicle or unit 16 which receives wet organic waste from said wet organic waste generator container,

a size reduction unit consisting of a grinding unit 21 which receives wet organic waste from said transportation and preparation unit and receives fluid from a primary fluid tank 30 to reduce said wet organic waste in size,

a density filtering unit 22 connected to said grinding unit and receives fluid from a secondary fluid tank 29,

a dryer unit 23 which receives portion of wet organic waste separated by said density filtering unit for further processing,

a mixing unit 24 which is connected to said density filtering unit and receives fluid from said secondary fluid tank to form a mixture,

an aerating unit 25 which is connected to said mixing unit for rapid organic solid reduction in mass by means of biological process,

a settling unit 26 which is connected to said aerating unit for separating fluid and organic solids for reuse or disposal,

a conditioning unit consisting of at least a dewatering unit 31.1 or wet organic compost unit 31.2 and connected to said settling unit,

a storage unit 80 connected to said settling unit and said conditioning unit.

Wet waste processing plant 20.2 is also preferred to be anaerobic such as controlled bioreactors or septic systems since most active landfills can easily be converted to a bioreactor to reduce initial capital outlay investment and associated cost.

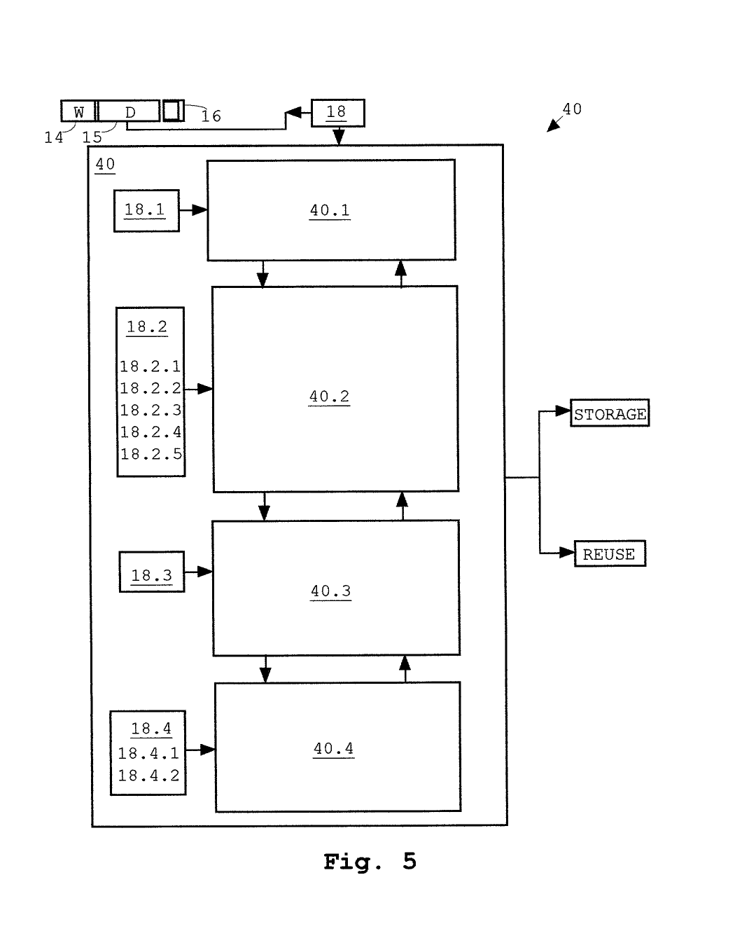

FIG. 5 shows a plan view of the present invention including dry waste processing facility or sub-system 40. Dry waste 18 is preferred to be separated into five primary groups of 18.1, 18.2, 18.3, and 18.4 for processing and reuse prior to landfill application.

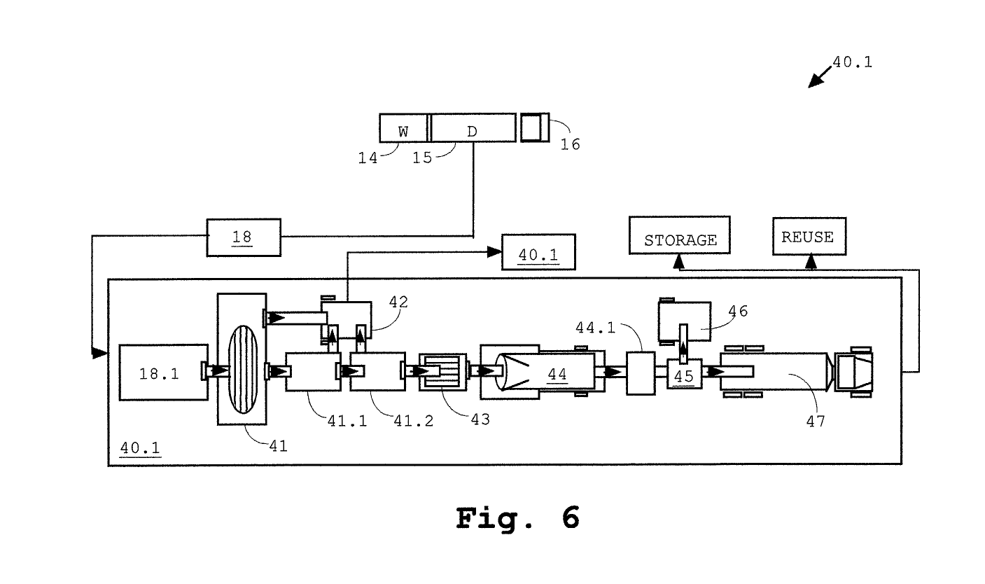

FIG. 6 shows a construction waste separation and processing unit 40.1 which receives construction waste 18.1. Cutting unit 41 breaks large pieces of dry waste 18.1 into smaller which then goes to construction waste segregation unit 41 where metals and plastics and wood are separated in mobile container 42 for further processing and remaining portion of said dry waste 18.1 is crushed using multi layer crushing unit 43 for further size reductions as desired and send to hammermill unit 44. Aggregate chips from said hammermill unit 44 is separated into different size products using multi screen 45 and send to mobile container 46 and 47 for reuse.

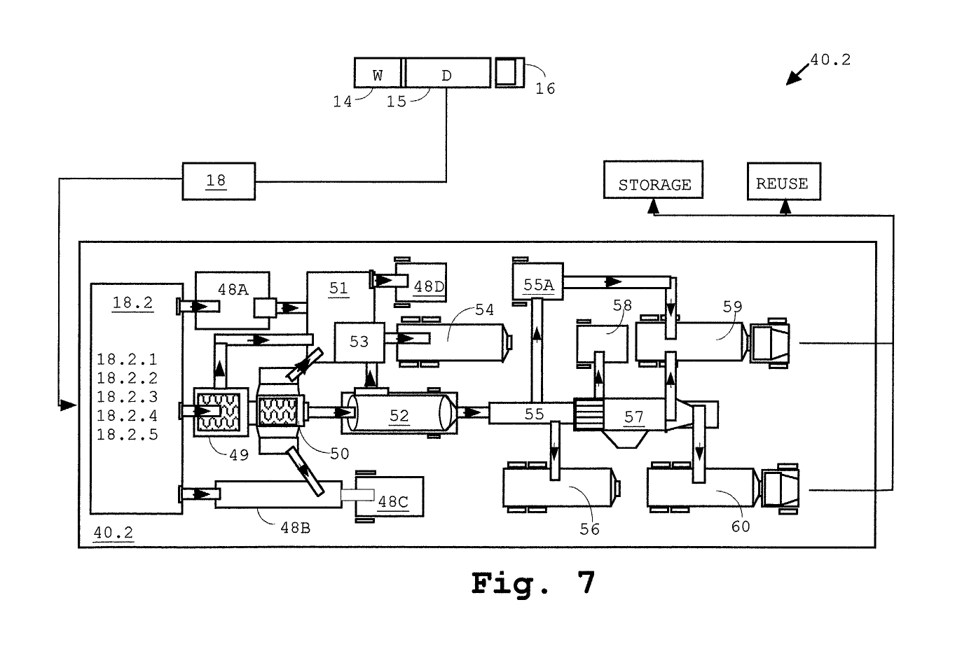

FIG. 7 shows a combined dry waste processing unit 40.2. Combined waste group 18.2 consist of at least one or more of aggregate waste 18.2.1 and plastic waste 18.2.2 and metal waste 18.2.3 such as ferrous and aluminum waste and glass waste 18.2.4 and white goods 18.2.5 and sub-groups are separated and processed using said combined dry waste processing unit 40.2. White goods 18.2.5 are separated as much as possible using separator 48B. Aggregate waste 18.2.1 is also separated as much as possible using 48B. Said combined waste 18.2 is accepted by primary grizzly dynamic screen 49 and secondary grizzly dynamic screen 50. Most of the loose aggregate portion of said combined waste is captured and separated by said primary and secondary grizzly dynamic screen 49 and 50 respectively and send to 48A from which collected aggregate waste is send to primary soil decontamination unit 51 which is connected to secondary soil decontamination unit 53. Said units 51 and 53 are to eliminate potential contaminants such as hydrocarbons and heavy metals. Remaining portion of said combined waste is accepted by multi screen trommel 52 through which all remaining aggregate waste is separated and send to secondary soil decontamination unit 53 and accepted by mobile storage unit 54 for reuse. All remaining portion of said combined waste passes through said trommel 52 and accepted by magnetic separator 55 which collects ferrous portion of metal waste which is accepted in mobile storage 56 for reuse. An air knife 57 separates plastics and non ferrous metal waste to be collected in mobile storage 60 and 59 respectively for reuse.

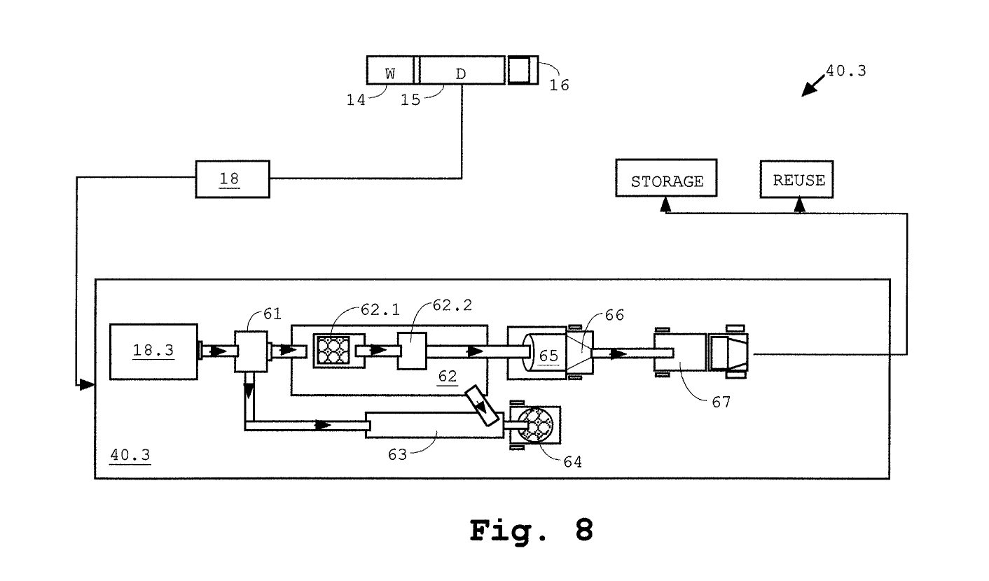

FIG. 8 shows an elastic waste separation and processing unit 40.3 which accepts wastes such as tire waste 18.3 and a portion of said tire waste is selected and contained in 64 for resurfacing or reuse as whole tire using tire preselection and post selection units 61 and 63 respectively. Processing unit 62 consist of prewash unit 62.1 and tire decontamination unit 62.2. Remaining portion of said tire waste is accepted by cross knife chipper 65 for further processing to be reduced to different sizes and collected in a mobile storage 67 for reuse.

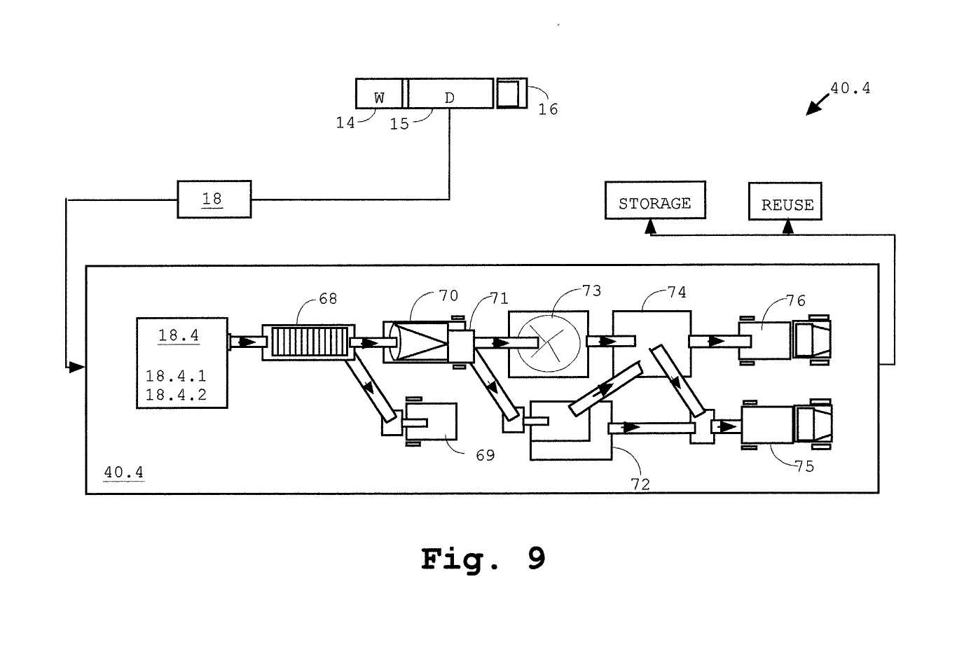

FIG. 9 shows a botanical waste separation and processing unit 40.4 which receives waste group 18.4 consisting of yard waste 18.4.1 such as grass clippings, tree trims, and wood waste 18.4.2 such as trees and wood from land clearing activities. All reusable wood is recovered using separation platform 68 and sent to a mobile storage 69. Remaining portion of the said waste group is sent to tree and multi-grade wood chipper 70 to be reduced to desired sizes and segregated using 71 for composting 73 or untreated mulch storage 72. Different grades of treated mulch from composting 73 is accepted by mobile storage 74 or 75 for reuse. In summary, preferred waste processing sub-system for dry waste portions of said waste includes

a transportation and preparation vehicle or unit 16 which receives and transports said dry waste from a dry waste container 18,

a construction waste separation and processing unit 40.1 which receives construction waste 18.1 portion of said dry waste from said transportation and preparation unit and process said construction waste into reusable materials for reuse,

a combined dry waste processing unit 40.2 which receives combined dry waste portion 18.2 of said dry waste including but not limited to aggregate waste and plastic waste and metal waste and glass waste and white goods from said transportation and preparation unit and process said combined waste into reusable materials for reuse,

an elastic waste separation and processing unit 40.3 which receives elastic waste portion 18.3 of said dry waste including but not limited to tires from said transportation and preparation unit and process said elastic waste into reusable materials for reuse, and

a botanical waste processing and separation unit 40.4 which receives botanical waste portion 18.4 of said dry waste including but not limited to yard waste and wood waste from said transportation and preparation unit and process said botanical waste into reusable materials for reuse.

While I have fully shown and described embodiments of my method and apparatus for integrated waste collection and management no limitations as to the scope of the present invention should be implied from the foregoing description. The true scope of the present invention is limited only by the following claims.

I claim:

1. Hydrotransportation of municipal waste comprising
a collection unit,
a transportation pipe connected to said collection unit, and
a wet waste processing plant which receives said wet waste from said transportation pipe and processes said wet organic waste using one or a combination of anaerobic and aerobic methods.

2. A collection unit as set forth in claim 1 includes one or a combination of
wet waste lift station connected to said transportation pipe,
wet waste transfer station connected to said transportation pipe.

3. A collection unit as set forth in claim 1 includes one or a combination of
a waste grinder connected to said collection unit,
a waste cutter connected to said collection unit.

4. Collection and processing of waste which is separated into wet organic waste and dry waste portions of said waste comprising
a collection apparatus which include one or a combination of
a wet organic waste generator container,
a dry waste generator container,
a transportation vehicle which includes at least one or a combination of
a wet organic transportation container attached to said transportation vehicle,
a dry waste transportation container attached to said transportation vehicle,
a wet organic waste processing plant which receives said wet organic waste and processes said wet organic waste using one or a combination of anaerobic and aerobic methods, and
a dry waste processing facility which receives said dry waste and processes said dry waste.

5. Waste collection apparatus as set forth in claim 4 includes a waste generator container comprising one or a combination of
a wet organic waste generator container which has a cover and contains said wet organic waste for collection,
a dry waste generator container which has a cover and contains said dry waste for collection.

6. Waste transportation vehicle for transportation of wet organic waste and dry waste portions of said waste comprising at least one or a combination of
wet organic waste transportation unit including a preparation and initial processing sub-unit for said wet organic waste during collection and transportation,
dry waste transportation unit including a preparation and initial processing sub-unit for said dry waste during collection and transportation.

7. Wet waste processing plant as set forth in claim 4 further comprises
a size reduction unit which receives wet organic waste from said transportation vehicle and receives fluid from a fluid tank to reduce said wet organic waste in size,
a mixing unit which is connected to said size reduction unit and receives wet organic waste from said reduction unit and fluid from said fluid tank to form a mixture,
an aerating unit which receives said mixture from said mixing unit for processing,
a settling unit which is connected to said aerating unit for separating fluid and organic solids for reuse or disposal.

8. Dry waste processing facility as set forth in claim 4 further comprises
a construction waste separation and processing unit which receives construction waste portion of said dry waste and process said construction waste into reusable materials for reuse,
a combined dry waste processing unit which receives combined dry waste portion of said dry waste including but not limited to aggregate waste and plastic waste and metal waste and glass waste and white goods and process said combined waste into reusable materials for reuse,
an elastic waste separation and processing unit which receives elastic waste portion of said dry waste including but not limited to tires and process said elastic waste into reusable materials for reuse, and
a botanical waste processing and separation unit which receives botanical waste portion of said dry waste including but not limited to yard waste and wood waste and process said botanical waste into reusable materials for reuse.

9. A wet organic waste generator container as set forth in claim 4 includes
at least one wheel connected to said wet organic waste generator container,
a removable cover connected to said wet organic waste generator container
a handle connected to said wet organic waste generator container, and
a removable liner which covers inside wall of said wet organic waste generator container and said liner is biodegradable.

10. A dry waste generator container as set forth in claim 4 includes
at least one wheel connected to said dry waste generator container,
a removable cover connected to said dry waste generator container,
a handle connected to said dry waste generator container, and
a removable liner which covers inside wall of said dry waste generator container.
